(19) United States
(12) Patent Application Publication
(10) Pub. No.: US 2007/0098809 A1
(43) Pub. Date: May 3, 2007

(54) Therapeutic use of lipids extracted from mother of pearl

(75) Inventors: Lopez Evelyne, Paris (FR); Elian Lati, Vanves (FR); Bernard Djian, Paris (FR)

Correspondence Address: NIXON & VANDERHYE, PC, 901 NORTH GLEBE ROAD, 11TH FLOOR, ARLINGTON, VA, 22203, US

(73) Assignee: SOCIETE D'INNOVATION ET DE RECHERCHE APPLIQUEE SA (SIERA SA), Paris (FR)

(21) Appl. No.: 10/561,237

(22) PCT Filed: Jun. 18, 2004
(86) PCT No.: PCT/FR04/01534

§ 371 Date: Apr. 5, 2006

(30) Foreign Application Priority Data
Jun. 20, 2003 (FR) 03/07509

(51) Int. Cl. A61K 31/22; A61K 35/56

(52) U.S. Cl. 424/547; 514/547

(57) ABSTRACT

The invention relates to the use of lipids extracted from the mother of pearl of mother-of-pearl molluscs, as a medicament, in particular in pathologies and traumas relating to a decrease in filaggrin activity and/or an increase in membrane transglutaminase activity. The invention also relates to pharmaceutical compositions containing same.

The invention relates to the use of lipids extracted from the mother of pearl of mother-of-pearl molluscs, as a medicament, in particular in pathologies and traumas related to a decrease in filaggrin activity and/or an increase in membrane transglutaminase activity. The invention also relates to pharmaceutical compositions containing said lipids.

Psoriasis is a chronic inflammatory skin disease which currently affects over 2million people in France and 3-5% of the population in Europe. With some 60,000 new cases diagnosed each year in France, it is one of the most frequent dermatological disorders. Although the cause has not yet been fully elucidated, today it is considered to be an autoimmune disease involving genetic and environmental factors.

The disease is characterized by erythematous plaques covered with thick whitish scales. Lesions can occur on the elbows, knees, lower back, scalp, feet, nails and in intertriginous areas. In the most severe cases, the ears, face, or even the entire body can be affected. While most of the time the condition is benign, it has the disadvantage of being an unsightly skin disease. Although not contagious, 3% of afflicted individuals are victims of all sorts of preconceived ideas. In about 8% of patients psoriasis is a serious disease, being generalized to the entire body or causing severe complications, in particular arthritic.

Psoriasis is characterized by (1) epidermal hyperproliferation (excessive growth with incomplete and accelerated differentiation) and (2) locally visible inflammation of the epidermis and dermis. It is currently thought that the disease is caused by and/or develops through activation of antigen-specific T lymphocytes, autoimmune reactions, and proliferation of mediators that induce keratinocyte proliferation. Thus, the expression stage of the disease is characterized by (1) an activation of T lymphocytes in particular due to gamma interferon, (2) an activation of epidermal cells (keratinocytes secrete inflammatory cytokines such as IL1, TNFα, and IL8, and chemokines), and (3) recruitment of endothelial cells in particular those of the venous capillary system leading to vasodilation and interaction with circulating leukocytes. Keratinocytes divide too rapidly according to a deregulated sequence of keratinization which leads to hyperproliferation of the epidermal layers and epidermal thickening. This manifests as hyperkeratosis or thickening of the horny layer which becomes dry and flaky.

In view of these multiple mechanisms, a number of treatments including various topical treatments, phototherapies, and oral treatments are in frequent use today to treat patients suffering from psoriasis. Topical treatments include in particular corticosteroids, coal tar, anthralin, vitamin D3and derivatives, retinoids, and UV radiation. The side effects of topical treatments include skin thickening, brown spots, burning, irritation and photosensitivity. Corticosteroids can also induce tolerance, making subsequent steroid-based therapies ineffective. Phototherapy comprises a medically supervised course of ultraviolet light therapy with UV B rays (UV-B) or treatment with psoralen combined with UV-A. Long-term phototherapy can cause premature skin ageing and an increased risk of skin cancers. Oral treatments, generally used in the most severe cases, comprise methotrexate, oral retinoids and cyclosporine. Therapy with methotrexate must be closely monitored to avoid hepatic damage. Oral retinoids must be used with care in women because of their teratogenic potential. Cyclosporine, an immunosuppressive drug, is restricted to patients who are refractory to other oral treatments or for whom other treatments are contra-indicated. Alternating these treatments, possibly in combination with phototherapy, has proved effective in some patients.

So far, then, no treatment is fully satisfactory and not all patients respond to or tolerate existing treatments. New alternative treatments of psoriasis are therefore being sought, in particular to improve efficacy and/or reduce side effects. Finally, a more optimal use of existing drugs in association with new treatments might lead to significant improvements.

Filaggrin is a protein located in the outermost layers of the epidermis. Profilaggrin, a histidine-rich protein, is the main component of the keratohyalin grains in the stratum granulosum. During terminal differentiation, profilaggrin is dephosphorylated and partially proteolyzed to intermediate compounds, then to filaggrin. Filaggrin promotes aggregation of keratin filaments by catalyzing the formation of disulfide bridges between them. It is one of the components of the horny envelope and plays a role in hydration since it is the main reservoir of natural moisturizing factors (NMF). In fact, the degradation of filaggrin provides a source of amino acids which are metabolized to NMF components, in particular glutamine is converted to PCA (hydrolidone carboxylic acid) and histidine to urocanic acid. Thus, low levels of NMF characterize major skin disorders, such as psoriasis (NMF levels practically nil), but also ichthyosis (NMF levels practically nil) and atopic dermatitis (NMF levels low relative to normal skin). The epidermal renewal time for a psoriasis patient is only 8days instead of approximately 21to 28days for normal epidermis. A phenomenon which tends to avoid this decrease in the levels of natural moisturizing factors is then observed, consisting of accelerated hydrolysis of filaggrin leading to the depletion of same.

In addition, psoriasis is characterized by very specific changes in some epidermal markers, in particular an increase in keratinocyte production of membrane transglutaminase (or transglutaminase 1) which plays a role in horny envelope formation. Thus, modifying this level would also make it possible to improve the condition of the skin in subjects afflicted with this disorder.

Rheumatoid arthritis is also an autoimmune disease involving genetic and environmental factors. This pathology is characterized by painful joint inflammation and is often accompanied by joint deformity. Many cases of rheumatoid arthritis are associated with anti-filaggrin autoantibodies. The commonly called “anti-keratin” antibodies are highly specific markers of this pathology. They are associated with the most severe forms of the disease and frequently precede the onset of clinical signs. They are synthesized by plasma cells in the synovial membrane. Said antibodies have been shown to recognize different molecular forms of profilaggrin and filaggrin. Today, specific autoantibodies directed against epidermal filaggrin (AFA for Anti-Filaggrin Antibodies) are considered the most important and reliable markers of rheumatoid arthritis.

In this context, the applicant has now demonstrated the interesting biological properties of lipids extracted from the mother of pearl of molluscs, namely the action of same on filaggrin and/or transglutaminase. In particular, it was shown that said lipid extracts from mother of pearl applied on the skin induce an overexpression of filaggrin and inhibit the expression of membrane transglutaminase.

The invention is therefore directed to a pharmaceutical composition comprising lipids extracted from the mother of pearl of mother-of-pearl molluscs in a pharmaceutically acceptable support.

The invention also deals with lipids extracted from the mother of pearl of mother-of-pearl molluscs as a medicament, in particular for treating pathologies in which there is a decrease in filaggrin activity and/or an increase in membrane transglutaminase activity, more specifically for treating pathologies related to a decrease in cutaneous filaggrin activity and/or cutaneous overexpression of membrane transglutaminase. In particular, the lipids extracted from the mother of pearl of mother-of-pearl molluscs can be used for treating skin pathologies, more particularly selected in the group consisting of psoriasis, ichthyosis, atopic dermatitis, but also autoimmune diseases linked to an autoimmune reaction to filaggrin, such as rheumatoid arthritis in particular.

In an altogether surprising manner, it was found that the mother of pearl from mother-of-pearl molluscs (molluscs capable of producing mother of pearl), in particular oysters and more specifically oysters of the genus Pinctada and more particularly of the genus Pinctada species margaritifera, contain lipids that promote skin regeneration. Furthermore, no intolerance was observed when said lipids were applied on human skin. In addition, as indicated earlier, said lipids extracted from mother of pearl and applied on the skin induce an overexpression of filaggrin and an inhibition of membrane transglutaminase expression.

More particularly, the mother of pearl lipids according to the invention are obtained by extraction from mother-of-pearl molluscs as defined hereinabove. The method of extraction more specifically comprises the following steps: 1) separating the mother of pearl from the rest of the shell of a mother-of-pearl mollusc, more particularly so as to obtain only the aragonite layer, 2) reducing the mother of pearl to a powder, 3) extracting the lipids from said powder by subjecting it to at least one extraction solvent or a mixture of solvents, then advantageously 4) extracting the lipids from the solvent or the solvent mixture used. Advantageously, the powder obtained in step 2) has a mean particle size (in number) of less than 20 μm, preferably approximately 8 μm. Particle size is determined by classical methods known to those skilled in the art, such as sieving and/or the laser particle sizing method.

The mother of pearl can be separated from the rest of the shell by any known method and in particular by milling. The mother of pearl can be ground in several steps, particularly in a first step by crushing, then optionally by different grinding techniques.

Advantageously, the mother of pearl can be decontaminated, particularly by decontaminating wash(es), for example in sodium hypochlorite solution, then by drying.

The mother of pearl can also be sterilized, advantageously after being ground, by any known method. In particular, sterilization is carried out by irradiation (gamma rays) or heat (dry or moist heat) (Atlan G., Delattre O., Berland S., Le Faou A., Nabias G., Cot D., Lopez E., Interface between bone and mother of pearl implants in sheep, Biomaterials, 1999, 20: 1017-1022; Balmain J., Hannoyer B., Lopez E., Fourier transform infrared spectroscopy (FITR) and X-Ray diffraction analyses of mineral and organic matrix during heating of mother of pearl (nacre) from the shell of the mollusc Pinctada maxima, J. Biomed. Mater. Res. Applied Mat., 1999, 48 (5): 749-754).

Lipid extraction can be carried out by any known method. Thus, for example, the powder is contacted with a lipid extraction solvent such as a chloroform/methanol mixture, or ethanol, or else hexane, the mixture is optionally stirred for 1 to 6 hours, then a separation is carried out, for example by centrifugation or filtration, to separate the solid phase from the liquid phase, advantageously followed by elimination of the solvent of the liquid phase, in particular by evaporation, to recover the desired lipids. In this way virtually all the desired lipids are recovered. According to a particular embodiment, the lipids used according to the invention, in particular extracted in this way, are not subjected to any chemical modification, particularly saponification, before, during or after extraction.

When the lipid extraction solvent is the chloroform/methanol mixture or hexane, contact, and optionally stirring, are advantageously carried out at room temperature (between approximately 18° C. and 25° C.). When the lipid extraction solvent is ethanol (absolute ethanol), contact, and optionally stirring, are advantageously carried out at a temperature comprised between 30° C. and 40° C., and preferably in powder/solvent weight ratios corresponding to 1/5. The weight ratios of the chloroform/methanol mixture are advantageously 2/1 and the powder/solvent weight ratio is preferably 1/3. When the lipid extraction solvent is hexane, the powder/solvent weight ratio is preferably 1/3.

When the lipids are separated from the solvent used, they are generally in the form of a brown paste which can be employed directly in the pharmaceutical composition according to the invention. Other separations can subsequently be carried out on the lipids obtained in this manner.

Thus, all or part of the lipids from mother of pearl, in particular obtained according to the method described hereinabove, can be put to use according to the invention.

The lipids so obtained correspond in particular to a mixture of polar and apolar compounds. Said mixture generally comprises ceramides, cholesterol, possibly cholesterol sulfate and/or acetate, fatty acids, triglycerides and a polar lipids (more a polar than the aforementioned lipids), as is more particularly the case for an oyster, advantageously an oyster of the genus Pinctada (generally harvested in its natural biotope). Of course, the nature and quantity of these compounds are subject to large variations, particularly as a function of the mollusc, the biotope of same, the season of harvest and the harvesting conditions.

Thus, to give a rough idea, the lipids extracted (with a 2:1 chloroform/methanol solvent mixture) from Pinctada margaritifera mother of pearl were examined by thin-layer chromatography after migration of the mobile phase hexane/diethyl ether/acetic acid on silica gel and coloration with copper sulfate. The percentages expressed as the intensity of coloration of the lipid compounds are given below:
Cholesterol sulfate: 0.5%
Hydroxylated ceramides: 2.07%
Nonhydroxylated ceramides: 1.03%
Cholesterol : 5.97%
Fatty acids: 6.24%
Triglycerides: 4.46%
Cholesterol acetate: 0.7%
A polar lipids (more a polar than the aforementioned lipids) : 79.03%
Omega was also detected among these lipids.

As shown in further detail in the experimental section to follow, the lipids obtained in this manner display very interesting properties. Said properties include those related to the effects on filaggrin and membrane transglutaminase noted hereinabove. Lipids extracted from mother-of-pearl molluscs, when applied on skin, also restore keratinocyte differentiation with a reduction in cell hyperproliferation. They also have the advantage of being completely harmless and of having an anti-inflammatory effect.

Thus, the invention has as object a pharmaceutical composition, characterized in that it comprises lipids such as defined hereinabove and a pharmaceutically acceptable excipient.

Administration of the inventive composition can be carried out by the topical route, and possibly enterally or parenterally. Preferably, the pharmaceutical composition is packaged in a form suited to topical application.

By the enteral route, the pharmaceutical composition can be in the form of tablets, capsules, lozenges, syrups, suspensions, solutions, powders, granules, emulsions, lipid or polymeric vesicles or microspheres or nanospheres allowing controlled release. By the parenteral route, the composition can be in the form of solutions or suspensions for infusion or injection.

According to a particular embodiment of the invention, the pharmaceutical composition is intended for topical use on the skin. Thus, the pharmaceutically acceptable support is in particular an excipient suited to topical application. By the topical route, the pharmaceutical composition according to the invention is more particularly intended for the treatment of the aforementioned pathologies, in particular for the treatment of the skin and mucous membranes. It can be in the form of salves, creams, milks, ointments, powders, saturated buffers, solutions, gels, sprays, lotions or suspensions. It can also be in the form of lipid or polymeric vesicles or microspheres or nanospheres or polymer patches or hydrogels allowing controlled release. Said topical composition can be in anhydrous form, in aqueous form or in the form of an emulsion (water/oil, oil/water or multiple emulsion).

The inventive lipids are used, preferably by the topical route, at a concentration generally comprised between 0.02% and 3% by weight, preferably between 0.25% and 2% by weight, and advantageously between 0.5% and 1% of the total weight of the composition.

In particular, the therapeutic composition according to the invention is characterized in that it contains, in combination with inactive excipients, a therapeutically effective amount of lipids extracted from the mother of pearl of mother-of-pearl molluscs such as defined hereinabove, particularly for treating pathologies involving a decrease in filaggrin activity and/or an increase in membrane transglutaminase activity, more specifically for treating pathologies related to a decrease in cutaneous filaggrin activity and/or cutaneous overexpression of membrane transglutaminase.

The invention is also based on a method of treatment of pathologies or traumas related to a decrease in filaggrin activity and/or an increase in membrane transglutaminase activity in subjects afflicted with said pathologies or traumas, comprising administering to said mammals a therapeutically effective amount of lipids extracted from mother of pearl such as defined hereinabove, in particular for treating pathologies related to a decrease in cutaneous filaggrin activity and/or cutaneous overexpression of membrane transglutaminase.

More specifically, the pathologies related to a decrease in cutaneous filaggrin activity and/or cutaneous overexpression of membrane transglutaminase are cutaneous pathologies, more particularly selected from psoriasis, ichthyosis and atopic dermatitis, and autoimmune diseases related to an autoimmune reaction to filaggrin, such as rheumatoid arthritis in particular.

In the spirit of the invention, the term “treatment” denotes preventive, curative, palliative treatment as well as management of patients (alleviating suffering, improving quality of life, slowing disease progression), and the like.

Furthermore, the treatment can be carried out in combination with other ingredients or treatments, in particular such as other compounds active in treating the aforementioned pathologies or traumas.

The other treatments can be those mentioned earlier, including a variety of topical treatments, phototherapies and oral treatments. lin particular, topical treatments include corticosteroids, coal tar, anthralin, vitamin D3 and derivatives, retinoids and UV rays. Phototherapy encompasses medically supervised treatment with type B ultraviolet rays (UV-B) or treatment with psoralen combined with UV-A. Oral treatments comprise administration of methotrexate, oral retinoids or cyclosporine.

The pharmaceutical compositions or medicaments according to the invention can also comprise at least one other therapeutically active ingredient, selected in particular from among those mentioned earlier, for use that is concurrent, separate or spread out over time, in particular during a treatment in a subject with a pathology or trauma related to a decrease in filaggrin activity and/or an increase in membrane transglutaminase activity, such as defined hereinabove.

The pharmaceutical compositions or mediaments according to the invention advantageously comprise one or more vehicles or excipients which are inert, that is to say, inactive and nontoxic. Examples include pharmaceutically compatible saline, physiologic, isotonic, buffered solutions and the like, known to those skilled in the art. The compositions can contain one or more agents or vehicles selected from among dispersives, solubilizers, stabilizers, preservatives, and the like. Agents or vehicles that can be used in the formulations (liquid and/or injectable and/or solid) comprise in particular methylcellulose, hydroxymethylcellulose, carboxymethylcellulose, cyclodextrins, polysorbate 80, mannitol, gelatin, lactose, vegetable or animal oils, acacia and the like. The compositions can be formulated as injectable suspensions, gels, oils, tablets, suppositories, powders, gelatin capsules, capsules, and the like, possibly by means of pharmaceutical forms or devices allowing sustained and/or delayed release. For this type of formulation, an agent such as cellulose, carbonates or starches is advantageously used.

It is understood that the aforementioned compositions can also contain pharmacodynamically active additives or a combination of said additives, and in particular: wetting agents; emollients; moisturizing agents, like glycerol, PEG 400 or else urea; other anti-psoriasis agents such as in particular corticosteroids, coal tar, anthralin, vitamin D3 and derivatives, and retinoids.

Said compositions can also contain flavor enhancers, preservatives such as para-hydroxybenzoic acid, stabilizers, moisture-regulating agents, pH-regulating agents, agents that modify osmotic pressure, emulsifiers, UV-A and UV-B filters, antioxidants, such as alpha-tocopherol, butylhydroxyanisole, or butylhydroxytoluene.

It is understood that the person of the art will take care to select any component(s) to be added to said compositions in such as way that the advantageous properties intrinsically related to the invention are not at all or not substantially modified by the planned addition.

Administration can be carried out by any method known to those skilled in the art, in particular by the oral or topical route or by injection, typically by the intraperitoneal, intracerebral, intrathecal, intravenous, intra-arterial or intramuscular route. Administration by the oral or topical route is preferred. In the case of a long-term and non-topical treatment, the preferred route of administration is sublingual, oral or transcutaneous.

For injections, the compounds are generally prepared in the form of liquid suspensions, which may be injected through syringes or by infusion, for instance. It is understood that the injection rate and/or injected dose, or generally speaking the dose to be administered, may be adapted by those skilled in the art according to the patient, the pathology, the mode of administration, etc. It is understood that repeated administrations may be given, possibly in combination with other active ingredients or any pharmaceutically acceptable vehicle (buffers, saline, isotonic solutions, in the presence of stabilizers, etc.).

According to a particular aspect, the invention relates to a device, in particular adapted to subcutaneous or percutaneous injection, comprising the lipids such as defined hereinabove and a physiologically acceptable excipient, which in particular is adapted to subcutaneous or percutaneous injection. In particular, said device can be in the form of syringes or infusions.

The invention can be used in mammals, particularly in humans.

Other aspects and advantages of the invention will become apparent in the following examples, which are given for purposes of illustration and not by way of limitation.

### EXAMPLES

This section demonstrates the efficacy of the lipids such as defined hereinabove.

1—Preparation of Lipids Extracted from Mother of Pearl

Extraction of the pellet with a chloroform/methanol mixture: 60 g of the powder obtained from mother of pearl of the mother-of-pearl oyster Pinctada margaritifera was mixed with 120 ml of a chloroform/methanol mixture (weight ratio : 2/1) and stirred at room temperature for about 4 hours, then filtered and evaporated to recover the lipids.

The lipids so obtained were incorporated in a cream base hereinbelow referred to as the excipient, to give the formulations P1 and P2 indicated hereinbelow.

2—Preparation of Explants and Delipidation

An ether/acetone mixture was applied on a defined zone of a skin sample. Fifteen explants were taken from said zone delipidated in this manner. Another 6 explants were taken from a non-delipidated zone.

The 15 delipidated explants were divided into 5 groups:
D (Delipidated skin Control, 6 explants)
DE (Delipidated skin+Excipient, 3 explants)
DP1 (Delipidated skin+Product 1, 3 explants)
DP2 (Delipidated skin+Product 2, 3 explants)

The 6 non-delipidated explants were divided into two groups: N0 (Normal skin at T0) and N3 (Normal skin at T3h).

3 —Products Tested

The following products were tested:

Base formulation:Excipient
P1 formulation containing 0.5% lipids by weight
P2 formulation containing 1.0% lipids by weight
   4—Application of Products

The test products were applied on the explants immediately after their preparation. The products were administered to groups DE, DP1 and DP2 by a 3-hour topical application (4 mg per explant). Groups N0, N3 and D were left untreated.

5—Histology

At time T0, immediately after delipidation, 3 explants were taken from groups N0and D0. Half was fixed in ordinary Bouin's fixative, the other half frozen. At time 3 hours after the start of treatment, 3 explants were taken from each group (N3, D3, DE, DP1 and DP2) and fixed in the same manner.

The histologic study consisted in:
Histologic analysis of stratum corneum by Masson trichrome staining.
Filaggrin labelling with anti-filaggrin monoclonal antibody (BTI Cliniscience ref. BT 576) Clone OKTB1 detected by immunofluorescence with a biotin/streptavidin amplifier system and nuclei counterstained with propidium iodide.
Labelling of membrane transglutaminase with anti-MTG monoclonal antibody (Harbor Products TEBU ref. 5003) clone B.C1 detected by immunofluorescence with a biotin/streptavidin amplifier system and nuclei counterstained with propidium iodide.

### 6—RESULTS

Morphologic Examination of Stratum Corneum after Masson Trichrome Staining:

Non-Delipidated Skin at T0 (N0):

The stratum corneum was quite compact, sparsely foliated, keratinized at the surface and at the base.

Delipidated Skin at T0 (D0):

The stratum corneum was more compact and more keratinized at the surface and at the base.

Normal Skin at 3 h (N3):

The stratum corneum was more or less compact, sparsely keratinized at the surface and at the base.

Delipidated Skin at 3 h (D3):

The stratum corneum was compact, markedly keratinized at the surface and at the base.

Delipidated Skin +Excipient (DE):

The stratum corneum was fairly foliated and slightly keratinized.

Delipidated Skin +0.5% Lipid Formulation (DP1):

The stratum corneum was slightly foliated, slightly keratinized at the surface and more pronounced at the base.

Delipidated Skin +1.0% Lipid Formulation (DP2):

The stratum corneum was more or less foliated, slightly keratinized at the surface and more pronounced at the base. Similar to P1.

Filaggrin Immunolabelling:

Non-Delipidated Skin at T0 (N0):

Strong, highly irregular labelling was seen on 6 to 7 cell layers with marked overexpression as compared with the normal.

Delipidated Skin at T0 (D0):

The labelling was similar to that seen with non-delipidated skin at T0, but it was present on a smaller number of cell layers with weak labelling in epidermal structures, as sometimes observed with very dry skin.

Normal Skin at T3h (N3):

Strong, very irregular labelling was seen on 3 to 7 cell layers with marked overexpression as compared with the normal. The labelling was identical to that of non-delipidated skin at T0.

Delipidated Skin at T3h (D3):

A variable labelling pattern was observed, generally on a smaller number of cell layers, which was much weaker and more irregular.

Delipidated Skin +Excipient (DE):

Labelling was irregular and was seen on 4 to 5 cell layers. It was slightly higher than on delipidated skin at T3h.

Delipidated Skin +0.5% Lipid Formulation (DP1) at 3h:

Strong, more or less regular labelling was seen on 6 to 7 cell layers with marked overexpression as compared with the normal. Labelling was similar to that seen with non-delipidated skin at T3h.

Delipidated skin +1.0% lipid formulation (DP2):

Labelling was similar to that seen with P1 and was present on the same number of cell layers.

Labelling of Membrane Transqlutaminase:

Non-Delipidated Skin at T0 (N0):

Labelling was very light, quite weak and irregular on 2 to 3 cell layers.

Delipidated Skin at T0 (D0):

Labelling was very light, more or less weak and irregular on 2 to 3 cell layers, similar to that of normal skin at T0.

Normal Skin at T3h (N3):

Labelling was light, quite clear and regular on 2 to 3 cell layers, similar to that of normal skin at T0.

Delipidated Skin at T3h (D3):

Labelling was very weak on 1 cell layer and often absent from the stratum corneum.

Delipidated Skin +Excipient (DE):

Labelling was fairly clear and more or less regular on 2 to 3 cell layers.

Delipidated Skin +0.5% Lipid Formulation (DP1):

Labelling was weak, more or less regular on 2 to 3 cell layers.

Delipidated Skin +1.0% Lipid Formulation (DP2):

There was almost no labelling in the stratum corneum.

General Morphology of the Stratum Corneum

At T0 on non-delipidated skin, the stratum corneum was quite compact and keratinized at the surface and at the base. It was more compact and keratinized on delipidated skin. This feature reveals a structure of dry skin.

At T3h on normal skin, the stratum corneum had a relatively foliated structure. It was still quite compact on delipidated skin. On delipidated skin treated with formulation P1, it was slightly less compact and more or less foliated. Formulation P2 gave results similar to formulation P1.

Filaggrin Expression

At T0 on normal and delipidated skin, filaggrin was highly overexpressed, with labelling present on 6 to 7 cell layers over the entire stratum corneum.

At T3h on delipidated skin, filaggrin labelling was weaker, very irregular and present on a highly variable number of cell layers. On normal skin, it was identical to that of skin at T0.

With the excipient, filaggrin expression was slightly higher than with untreated delipidated skin at T3h.

Formulation P1 containing 0.5% lipids from mother of pearl induced an overexpression of filaggrin, present on 6 to 7 cell layers at an intensity greater than that seen on normal skin at T3h.

Formulation P2 containing 1% lipids from mother of pearl induced a very marked overexpression of filaggrin which was slightly greater than that seen with formulation P1.

Expression of Membrane Transglutaminase

At T0 on normal and delipidated skin, membrane transglutaminase expression was very low, highly irregular and present on 2 to 3 cell layers.

At T3h on delipidated skin, membrane transglutaminase (MTG) labelling was very weak and present on 1 to 2 cell layers. It was slightly greater in normal skin than in skin at T0. With the excipient, MTG expression was more marked than on normal and untreated delipidated skin.

Formulation P1 containing 0.5% lipids from mother of pearl induced very low MTG expression, much less than that seen with delipidated skin at T3h and skin treated with excipient.

Formulation P2 containing 1% lipids from mother of pearl induced practically no expression of MTG.

### CONCLUSION

The proteolysis of filaggrin produces, among other components, a large number of amino acids which enter into the composition of NMF (Natural Moisturizing Factors), located between the corneocytes of the stratum corneum. Filaggrin overexpression reveals the dryness of the skin samples used. This feature was amplified by delipidation. On delipidated skin, formulations P1 and P2 induced filaggrin expression to a level similar to that of normal skin. They supplied the stratum corneum with the components necessary for rapid reconstruction of the intercellular cement.

Both formulations P1 and P2 were active in reconstituting the intercellular cement, with formulation P2 containing 1% lipids from mother of pearl showing slightly more activity.

The composition of the horny envelope is controlled by membrane transglutaminase. The low MTG expression observed at T0 is characteristic of dry skin and was amplified by delipidation of the sample with the solvent mixture, thereby amplifying the alterations in the horny envelope. The skin therefore drew on its components reconstituting the horny envelope structure: MTG. Both formulations P1 and P2 appeared to inhibit MTG activity in a dose-dependent manner and to prevent normalization of MTG levels.

Both formulations P1 and P2 inhibited the expression of membrane transglutaminase.

1-14. (canceled)

15. A pharmaceutical composition comprising lipids extracted from the mother of pearl from mother-of-pearl molluscs in a pharmaceutically acceptable support.

16. The composition according to claim 15, wherein the mother-of-pearl molluscs are oysters.

17. The composition according to claim 16, wherein oysters are oysters of the genus Pinctada and more particularly of the genus Pinctada species margaritifera.

18. The composition according to claim 15, wherein the pharmaceutically acceptable support is an excipient suitable for topical, enteral or parenteral application.

19. The composition according to claim 15, wherein the pharmaceutically acceptable support is an excipient suitable for topical application.

20. The composition according to claim 15, wherein the lipids are present at a concentration comprised between 0.02% and 3% by weight, preferably between 0.25% and 2% by weight, and advantageously between 0.5% and 1%, relative to the total weight of the composition.

21. The composition according to claim 15, for the treatment of pathologies involving a decrease in filaggrin activity and/or an increase in membrane transglutaminase activity.

22. The composition according to claim 15, for the treatment of pathologies related to a decrease in cutaneous filaggrin activity and/or cutaneous overexpression of membrane transglutaminase.

23. The composition according to claim 15, for the treatment of cutaneous pathologies, more particularly selected in the group consisting of psoriasis, ichthyosis and atopic dermatitis.

24. The composition according to claim 15, for the treatment of autoimmune diseases related to an autoimmune reaction to filaggrin, in particular rheumatoid arthritis.

25. The composition according to claim 15, wherein it further comprises at least one other therapeutically active ingredient for use that is simultaneous, separate or spread out over time.

26. The composition according to claim 16, wherein the other therapeutically active ingredient is selected from corticosteroids, coal tar, anthralin, vitamin D3 and derivatives, and retinoids.

27. A device comprising lipids such as defined in claim 15 and a physiologically acceptable excipient.

28. The device according to claim 27, having a form which is suitable to subcutaneous or percutaneous injection, in particular in the form of a syringe or infusion.
